(19) United States
(12) Patent Application Publication
(10) Pub. No.: US 2022/0093267 A1
(43) Pub. Date: Mar. 24, 2022

(54) NONINVASIVE REAL-TIME PATIENT-SPECIFIC ASSESSMENT OF STROKE SEVERITY

(71) Applicants: Arizona Board of Regents on Behalf of the University of Arizona, Tucson, AZ (US); The University of Pittsburgh - Of the Commonwealth System of Higher Education, Pittsburgh, PA (US)

(72) Inventors: Kaveh LAKSARI, Tucson, AZ (US); Hessam BABAEE, Pittsburgh, PA (US)

(21) Appl. No.: 17/424,953

(22) PCT Filed: Jan. 22, 2020
(86) PCT No.: PCT/US2020/014604

§ 371 Date: Jul. 22, 2021

Related U.S. Application Data
(60) Provisional application No. 62/795,170, filed on Jan. 22, 2019.

(51) Int. Cl. G16H 50/50; A61B 6/00; A61B 6/03; A61B 8/06; A61B 8/08; A61B 5/055; A61B 5/00; G16H 30/40; G16H 50/30; G16H 50/20

(52) U.S. Cl. CPC G16H 50/50; A61B 6/504; A61B 6/032; A61B 6/501; A61B 8/06; G16H 50/20; A61B 8/0891; A61B 5/055; A61B 5/0042; G16H 30/40; G16H 50/30; A61B 8/488

(57) ABSTRACT

A method for obtaining information for use in determining stroke severity includes simulating, with a processor executing instructions stored on a compute readable medium, cerebral blood flow (CBF) using as inputs information extracted from an angiography scan of the stroke patient and information concerning blood flow in cranial arteries. Cerebral tissue viability of the stroke patient is assessed based at least in part on the simulated CBF.

### BACKGROUND

Stroke is the second leading cause of death and the third leading cause of disability worldwide. Current estimates indicate that a stroke occurs every 40 seconds in the U.S., leading to a stroke-related death every 4 minutes. Ischemic strokes, which are caused by blockage or narrowing of the vessel lumen, constitute approximately 90% of all strokes, where the formation of a thrombus on the blood vessel wall disrupts the blood flow patterns downstream of the thrombus, potentially leaving regions of the brain underperfused. Cerebral blood flow (CBF) is one of the most important parameters related to brain physiology and function. However, despite its importance, estimating regional CBF inside the skull is no trivial task. Imaging techniques that provide regional CBF measures may be unavailable due to time constraints or access to facilities. As well, current modeling approaches have proven inadequate and only provide averaged lumped values for the entire brain, due to the prohibitive size of the computation. Hence, a significant clinical need exists for the development of a prediction tool that incorporates patient-specific factors and provides three-dimensional (3D) maps of cerebral perfusion and other predictive hemodynamic measures in a timely manner.

### BRIEF DESCRIPTION OF THE DRAWINGS

FIG. 1A shows an example of a CTA stack and FIG. 1B shows a top view of extracted cerebral arteries from a stroke patient; FIGS. 1C and 1D shows a reconstruction (centerlines and local diameters) of a segment of brain vasculature including middle cerebral arteries (MCA), anterior cerebral arteries (ACA) and internal carotid arteries (ICA).

FIGS. 2A-2D shows CT perfusion scans from a patient with middle cerebral artery (MCA) occlusion, where FIG. 2A shows the cerebral blood flow, FIG. 2B shows the cerebral blood volume, FIG. 2C shows the mean transit time and FIG. 2D shows the time to peak.

FIGS. 3A-3D illustrate the overall process described herein, where FIG. 3A shows the geometry of the cerebral vasculature reconstructed from computed tomography angiography images. FIG. 3B shows the patient-specific cerebral blood perfusion simulated in real-time, FIG. 3C show simulations validated against perfusion imaging measurement and FIG. 3D shows stroke severity estimators that are developed based on a machine learning approach.

FIG. 4 is functional block diagram illustrating one example of a system for modeling and predicting stroke severity.

### SUMMARY

Described herein are techniques that build and validate a simulation and classification algorithm to estimate regional cerebral hemodynamics in ischemic stroke and provide tissue viability measures. Using routinely-performed computed-tomography angiography (CTA) images or similar angiography techniques, our model produces perfusion maps with significantly superior spatiotemporal resolution for the entire brain, which are equivalent to CT perfusion (CTP) images without the need for actual perfusion scanning. Contrary to currently available techniques, our model does not require any patient-dependent calibration or parameter optimization and exploits parallel computing to provide real-time estimates. Furthermore, using a new machine learning algorithm, we provide prediction measures for differentiating infarct tissue from salvageable penumbra. We validate our results against an existing clinical dataset of previous stroke patients. The final product is real-time, non-invasive, patient-specific estimates for brain tissue perfusion and viability, which we hypothesize can improve upon current threshold-based prediction measures. Among other things, we are able to (1) simulate patient-specific regional CBF for stroke patients and (ii) determine biofidelic predictors for infarcted core and penumbra using machine learning.

Regarding the simulation of patient-specific regional CBF for stroke patients, we reconstruct subject-specific vascular geometries based on each patient's CTA, and subsequently simulate regional cerebral hemodynamics in real time, leveraging our high-performance computational model. We validate our results against each subject's perfusion measurements from the same dataset. This result provides a database of time-dependent 3D brain perfusion parameters, including regional CBF, which we use to build our machine learning training set. Producing these results in real time is crucial in their potential clinical adoption, especially in the absence of perfusion imaging capabilities.

Regarding the determination of biofidelic predictors for infarcted core and penumbra using machine learning, we incorporate continuous time-dependent volumetric perfusion maps from hemodynamic simulations above to build a probabilistic classifier for infarct and penumbra tissue regions. Using Binary Gaussian Process (BGP) machine learning approach, we overcome traditional limitations of current threshold-based predictors. In addition to perfusion parameters, we include non-hemodynamic factors such as NIH Stroke Scale (NIHSS), age, history of diabetes and hypertension, in order to improve our model's ability to provide accurate predictions. We calculate certificates of fidelity for each predictor, with an aim to ensure the clinicians are confident in incorporating the predictor results in conjunction with their clinical intuition.

The techniques described herein present significant refinement over existing modeling approaches to quantifying stroke severity and tissue viability, given our emphasis on validation and clinical translation. Here, an innovative and rigorous hybrid medical imaging and computational approach is employed that can provide critical perfusion maps and risk estimates for tissue viability, without the need for perfusion scanning.

In one particular aspect, we have developed an algorithm that, given a sequence of CTA images, can automatically reconstruct 3D vessel geometries. First, we apply a “vessel-enhancement” filter to calculate intensity and curvature in each voxel. By applying a multi-scale second-order Gaussian smoothing filter, we improve signal to noise ratio in the multiple length scales of the cerebrovascular network. We then segment the vessels, using a method such as Chan-Vese active contours level-set algorithm, to achieve a high level of approximation. Spanning the length scales from largest vessels (cranial arteries) to vessels with diameters as small as the CTA resolution, we calculate each vessel's diameter, length, and tortuosity, and a connectivity matrix describing the branching patterns. This algorithm calculates the vesselness probability map (local tubularity). Finally, on the binary images, we perform a skeletonization algorithm to determine the vessel centerlines (3D skeletonization), local diameters at each point on the centerline, and the branching connections at each vessel bifurcation. The result is a 3D map of vessel centerline and corresponding diameter for the entire brain vasculature, from the large cranial vessels down to vessels with diameters at the level of CTA image resolution. Visualizing the 3D vascular distribution and architecture alone will be also helpful in highlighting vessel blockage sites for clinical use. We then use these patient-specific vascular geometries, including each branch's length, diameter, angle, and branching structure, as input to the computational fluid dynamic (CFD) model for blood flow simulations.

The entire brain vasculature contains order of millions of vessels. Simulating blood circulation in this massive network of vessels requires significant computational capability—particularly if time-urgent predictions are needed. A fast CFD model for blood flow simulation in the brain is developed that enables near real-time prediction of brain hemodynamics. We can achieve the desired computational efficiency with a three-pronged approach: (1) Modeling: we develop and utilize one-dimensional computational models that are orders of magnitude faster than their three-dimensional counterparts. This will trade tolerable modeling uncertainty for computational speed. (2) High Performance Computing: We can solve our one-dimensional model with parallel computers. For example, we use distributed computing, in which the entire brain vasculature is decomposed to non-overlapping domains each assigned to a processor that collectively solve the model. (3) High-Fidelity Numerical Methods: We discretize our continuous one-dimensional model using high-accuracy numerical algorithms, such as the discontinuous Galerkin method. For a desired level of accuracy, these high-fidelity numerical methods will lead to small-size discrete problems that are faster and more reliable to solve on computers.

The simulation of cerebral blood flow in the brain using the CFD model requires data concerning inlet boundary conditions at the cranial arteries and outflow boundary conditions for vessels. These boundary conditions may be patient-specific boundary conditions, or they may be specified generically for all patients or specific classes of patients. In the case of patient specific boundary conditions, a neck collar may be employed, instrumented with a non-invasive sensor (e.g., vascular Doppler ultrasound or plethysmograph) capable of measuring blood flow velocity/pressure at the level of large cranial arteries. Using these measurements as input to the computational model, together with the patient's CTA scans, the device will provide 3D estimate maps and confidence levels for infarct and salvageable tissue and will provide outcome measure for reperfusion surgery based on the machine learning algorithm.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Furthermore, the claimed subject matter is not limited to implementations that solve any or all disadvantages noted in any part of this disclosure.

### DETAILED DESCRIPTION

Stroke is the second leading cause of death and the third leading cause of disability worldwide. In the U.S., alone, stroke is estimated to afflict approximately 800,000 Americans each year, occurring every 40 seconds and leading to a stroke-related death every 4 minutes. Over 85% of all strokes are ischemic and occur either due to the narrowing or blockage of cerebral arteries caused by disease (thrombotic), or due to the migration of a clot from outside the cerebral vasculature into the arteries of the brain (embolic). Whichever the case, a complete blockage or narrowing of the blood vessels (stenosis) disrupts the cerebral blood flow (CBF) and reduces the blood supply to cells that are downstream of the thrombus. A reduction in the CBF below critical thresholds discriminates between salvageable penumbra and irreversible infarct core. The mantra “Time lost is brain lost” emphasizes the rate of nervous tissue loss post-stroke—1.9 million brain cells lost every minute—and the need for immediate diagnosis and therapeutic interventions.

A crucial decision in stroke patient management is the course of treatment to be prescribed. Specifically, depending on the stroke severity, a clinician must determine whether a patient should receive a potentially risky and costly thrombolytic intervention, or endovascular procedure and clot retrieval (mechanical thrombectomy). Recently concluded clinical trials on endovascular therapy for ischemic stroke found favorable outcomes based on measures of tissue perfusion, rendering CBF the most reliable measure related to brain physiology and function, as well as clinical outcomes. However, a major setback in acquiring CT perfusion (CTP) scans is the substantial gap in the availability of equipment and facilities, given the high initial capital equipment investments and procedural costs, which have proven prohibitive for smaller hospitals and clinics. In addition, there are several other drawbacks to these techniques, including the invasive nature of injecting exogenous contrast agents, and the need for high radiation exposure dosages to ensure high-fidelity image acquisition, which is a noted concern of the FDA. Further, the practical challenge is understanding the multiple facets of these imaging techniques by the physicians, in addition to managing the constraints of time, cost, access, preferences of treating physicians, and availability of expertise. A quantitative clinical tool that, despite the above hurdles, incorporates direct patient-specific factors such as cerebrovascular architecture and regional CBF, is critical for clinicians and the subsequent care for stroke patients in an emergency setting.

Unlike perfusion imaging, CT angiography (CTA) measurement is routinely performed in most hospitals in patients with a suspected ischemic stroke, according to guidelines published by American Heart Association (AHA) and American Stroke Association (ASA) Stroke Council. The techniques described herein take advantage of this opportunity: we build and validate a computational algorithm to extract the cerebrovascular architecture from CTA images, and, through simulation, produce CTP-equivalent 3D maps for each patient, without the need for perfusion scanners or protocols, or the other drawbacks identified above. Such an algorithm can be used in community hospitals and mobile emergency units that lack perfusion imaging capabilities to triage patients that might be a thrombectomy candidate, who could then be sent to a comprehensive stroke center capable of performing the necessary procedures.

There exists a large body of literature on computational modeling for cerebral blood flow. However, to date, due to challenges in model parameterization and computational costs, these models have mostly been confined to academic circles and their clinical adoption has been limited. Pre-existing vascular redundancies, such as the circle of Willis at the base of brain, or the neovascularization between adjacent blood vessels (anastomosis), have proven indispensable in allowing collateral flow and covering the deficiency of blood delivery due to blocked arteries. However, patient-specific modeling (incorporating the correct vascular tree geometry, collateral flow, vessel diameter, and tortuosity) is a relatively new phenomenon inclinical modeling. Most previous models are limited to population-averaged variables, restricting feasibility for clinical predictions.

Using a vessel reconstruction algorithm described herein we reconstruct each patient's vasculature from their own CTA images to provide accurate geometrical measures for simulation. Furthermore, computational cost has presented a prohibitive hurdle in simulating the entire brain given the enormous complexity of the cerebrovascular network. Real-time simulation of the entire cerebrovascular system is critical for clinical adoption of computational predictive tools, especially for stroke patients. Here, we use a previously developed one-dimensional (1D) computational model of the blood flow described in P. Perdikaris, L. Grinberg, and G. E. Karniadakis, “An Effective Fractal-Tree Closure Model for Simulating Blood Flow in Large Arterial Networks,” Annals of Biomedical Engineering, vol. 43, no. 6, pp. 1432-1442, 2015. By accounting for the viscoelastic compliance of the arterial walls, this computational fluid dynamic (CFD) model has an important advantage over other existing computational models that assume vessel walls to be rigid.

Having further developed the model by including the equations of perfusion, we can now simulate cerebral hemodynamics and produce results that are directly comparable with perfusion measurements from medical imaging. Our model is capable of performing real-time simulation for two primary reasons. First, it is highly parallelized (linearly scalable to up to 10,000 processors), thereby reducing the computational cost, which is inversely proportional to the number of processors. Secondly, our numerical discretization is based on a high-order Discontinuous-Galerkin method that requires an order of magnitude fewer degrees of freedom (DOF) compared to low order numerical methods. These capabilities allow us to perform the enormous computation in near real time, which has rendered other computational models more of a scientific rather than a clinical interest.

Despite the significant clinical need for a predictive tool, precisely identifying blood flow patterns, infarct core and salvageable ischemic penumbra remains a challenge for acute interventions. In accordance with the techniques described herein, a new hybrid medical imaging and computational approach is provided that utilizes high-performance computing and machine learning to provide measures of regional CBF and stroke severity. We developed and validated a simulation algorithm that provides real-time and patient-specific estimates for whole brain tissue perfusion and stroke severity without the need for costly perfusion imaging. This allows the access to the crucial 3D perfusion information to a much larger group of clinical settings, which may not have access to perfusion imaging technology.

Overlooking patient-specific information dramatically reduces the reliability of current models, as they invariably require calibration or tuning of parameters that relate the measured quantities to a particular and limited training set. The techniques described herein employ a fast vascular reconstruction algorithm that can take each patient's stack of CTA images and build the 3D vascular geometry for the entire brain in a matter of seconds

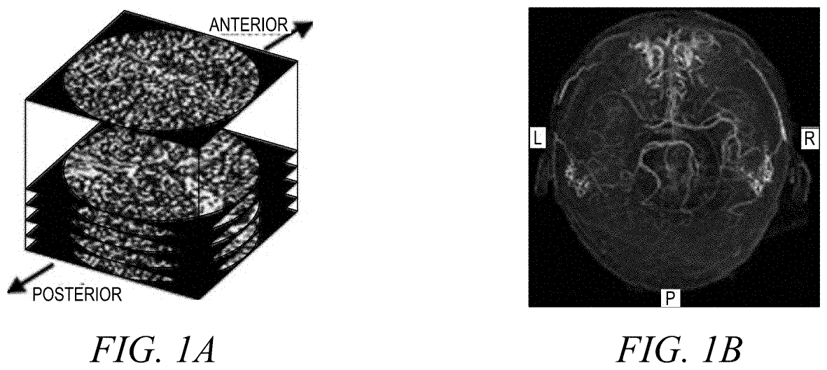

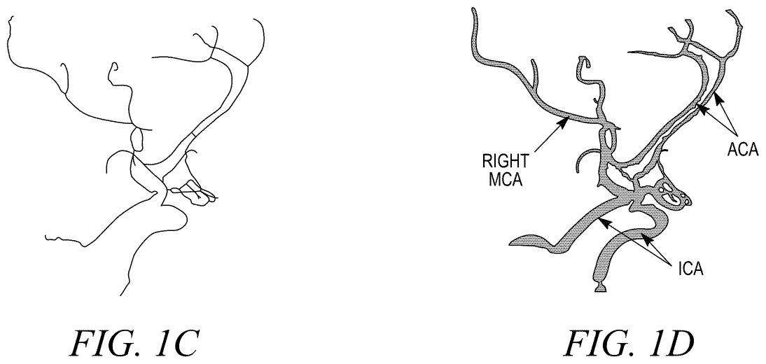

FIGS. 1A-1D illustrate the overall process of reconstructing the 3D vascular geometry from the CTA data. FIG. 1A shows an example of a CTA stack and FIG. 1B shows a top view of extracted cerebral arteries from a stroke patient. FIGS. 1C and 1D shows a reconstruction (centerlines and local diameters) of a segment of brain vasculature including middle cerebral arteries (MCA), anterior cerebral arteries (ACA) and internal carotid arteries (ICA). By employing patient-specific information in this manner, we ensure that variations in the vascular architecture of each patient is taken into account, and contrary to currently available models, avoid any patient-dependent calibration or parameter optimization. Furthermore, adding modeling capabilities to calculate blood perfusion in the tissue to a previously developed CFD model of blood flow, we can now simulate blood flow and produce 3D cerebral brain perfusion maps for each patient in real time, by scaling the computation to thousands of processors.

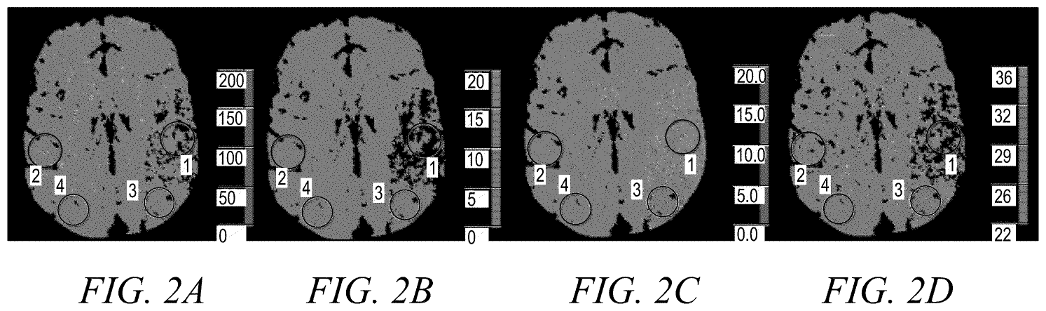

An important aspect of computational modeling is validation against experimental measurements. We have validated our modeling predictions, including CBF, cerebral blood volume (CBV), mean transit time (MTT), and time to peak (TTP), against a large dataset of previous stroke patients. An example of such data for a particular patient is shown in FIGS. 2A-2D, which shows CT perfusion scans from a patient with middle cerebral artery (MCA) occlusion. FIG. 2A shows the cerebral blood flow, FIG. 2B shows the cerebral blood volume, FIG. 2C shows the mean transit time and FIG. 2D shows the time to peak. This validation step allows the techniques described herein to provide accurate estimates for tissue perfusion that can be relied upon for clinical use. Furthermore, imaging modalities use complex, and often proprietary, mathematical convolutions to infer perfusion from parameters such as CBF and MTT. These mathematical approximations have been shown to widely vary between different companies for the same dataset, making them less reproducible and less reliable. Through our approach to uncertainty quantification in our machine learning algorithms and by providing certificates of fidelity, we ensure that the clinicians are confident in incorporating our predictions in conjunction with their clinical intuition.

The technology described herein can be eventually translated to clinics to help healthcare professionals more successfully identify and treat stroke patients. Currently, few software and algorithms exist for stroke severity assessment, e.g. RAPID (iSchemaView, CA) and AI-CT (Infervision, Inc.), all of which still rely on perfusion scans in order to make predictions. Our algorithm produces perfusion-equivalent estimates for the entire brain without the need for perfusion scans. This could potentially have immense effects, as this model can be adopted by clinics that lack perfusion scanning capabilities. Furthermore, this algorithm can be adopted by mobile emergency units or ambulances, which can analyze the patient's stroke severity while transferring the patient to the hospital.

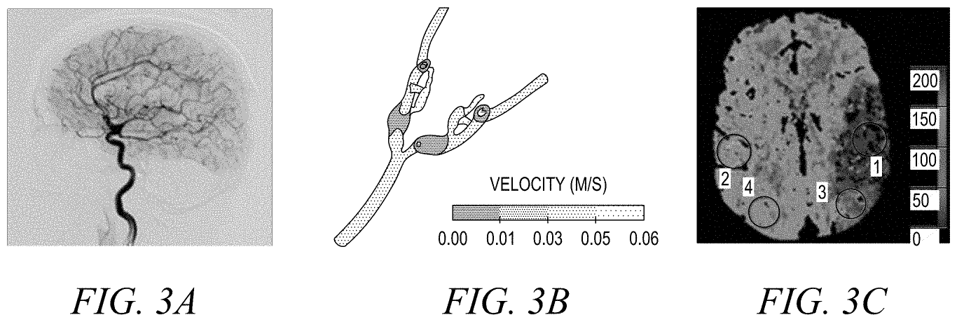

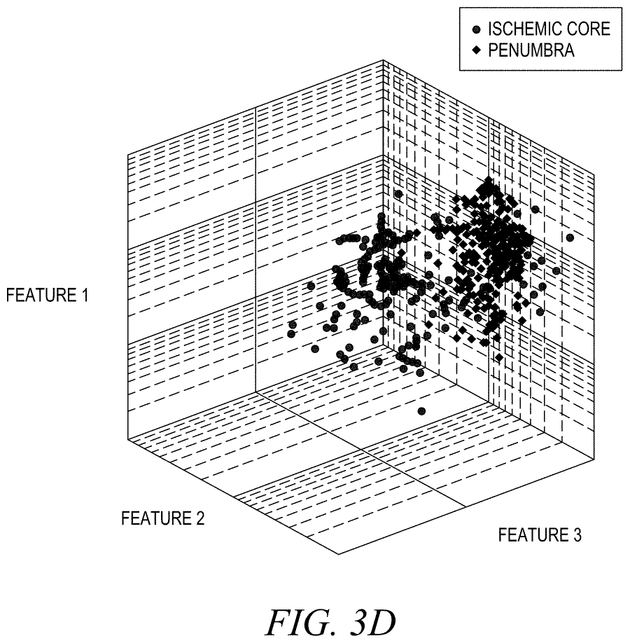

FIGS. 3A-3D illustrate the overall process described herein. More specifically, FIG. 3A shows the geometry of the cerebral vasculature reconstructed from computed tomography angiography images. FIG. 3B shows the patient-specific cerebral blood perfusion simulated in real-time. FIG. 3C show simulations validated against perfusion imaging measurement. FIG. 3D shows stroke severity estimators that are developed based on a machine learning approach. This process will be described in the following sections in more detail.

Section 1 describe mathematical methods that are used to determine the vascular geometry of each subject using CTA and CBF imaging datasets from stroke patients. We then simulate CBF patterns and validate our model against the same dataset. In Section 2, we use the validated model to determine the most predictive perfusion parameters for stroke severity assessment. Model components that influence this assessment are targeted to provide a physical understanding of how collateral flow could influence patient outcomes. The final products are 3D reconstructed vascular geometries and 3D perfusion maps (CBF, CBV, MTT, TTP) for each patient, and a machine learning scoring algorithm to assess the severity of stroke and delineate infarct and penumbra regions.

Section 1: Simulate Patient-Specific Regional CBF from Stroke Patients

The wide variation in vascular geometries and the development of collateral flow has limited clinical use of current population-averaged computational models, which calls for incorporating patient-specific information in hemodynamic simulations. In this Section we reconstruct subject-specific vascular geometries based on each patient's CTA, and subsequently simulate regional cerebral hemodynamics in real-time, leveraging our high-performance computing 1D model. This Section provides space- and time-dependent 3D maps of brain tissue perfusion, which can then be validated against our clinical data. Since CTA measurements are routinely performed on patients suspected of ischemic stroke, according to AHA guide-lines, by providing 3D vessel geometry maps, our vessel reconstruction techniques can be readily translated to clinical settings. Furthermore, since CTP measurements are not routinely done due to lack of access to scanning equipment or protocols, our blood flow perfusion simulations can provide perfusion-equivalent estimates, without the need for perfusion scans and its consequent exposure to radiation.

Automatic reconstruction of cerebral arteries is performed using an algorithm for reconstructing each patient's 3D vessel geometries. First, based on the Hessian-based approach described in A. F. Frangi, W. J. Niessen, K. L. Vincken, and M. A. Viergever, “Multiscale vessel enhancement filtering,” vol. 1496, pp. 130-137, 1998, we apply a “vessel-enhancement” filter to calculate intensity and curvature in each voxel. By applying a multi-scale second-order Gaussian smoothing filter, we improve signal to noise ratio in the multiple length scales of the cerebrovascular network. We then segment the vessels using a level-set algorithm to achieve a high level of approximation described in T. F. Chan and L. A. Vese, “Active Contours Without Edges,” IEEE Transactions on Image Processing, vol. 10, no. 2, pp. 266-277, 2001. Finally, we perform a skeletonization algorithm to determine the vessel centerlines and local diameters. The result is a 3D map of vessel centerline and corresponding diameter for the entire brain vasculature, from the large cranial vessels down to vessels with diameters at the level of CTA image resolution (0.6 mm). Visualizing the 3D vascular distribution and architecture is helpful in highlighting vessel blockage sites for clinical use. We use these patient-specific vascular geometries, including each branch's length, diameter, angle, and branching structure, as input to the CFD model described below.

Next, we simulate cerebral hemodynamics based on patient-specific CTA measurements. By assuming a specific branching pattern for the microvasculature based on Murray's law, we can overcome the cut-off radius sensitivities reported in other models. Having set the correct boundary conditions, we set out to simulate blood flow in the entire cerebral vasculature for each patient from a pool of previous stroke patients, which provides a dataset of time-dependent and space-dependent CBF, CBV, MTT, and TTP measures for each patient.

We use the images from our stroke patient cohort as ground truth and validate our simulation results against each patient's corresponding perfusion images. We use Bland-Altman plots of overall estimation performance for the numerical results compared with the corresponding imaging measurements. Our goal is to reach an approximation within one standard deviation for every pixel in the perfusion images. Although the performance of the 1D models has been previously demonstrated, disregarding radial variations in the blood flow could lead to discrepancies between the 1D simulations and the actual 3D flow. If that is the case when comparing our results to clinical perfusion images, we can use 3D simulations in the open source NekTar software (Y. Yu, H. Baek, and G. E. Karniadakis, “Generalized fictitious methods for fluid-structure interactions: Anal-ysis and simulations,” Journal of Computational Physics, vol. 245, pp. 317-346, 2013) to calibrate resistive parameters in the 1D models to better approximate blood flow properties.

### Section 2: Determine Biofidelic Predictors for Infarcted Core and Penumbra Using Machine Learning

Traditionally, simple thresholds have been used to categorize perfusion images into infarct core or salvageable penumbra depending on reperfusion status. However, this over-simplified approach of binary classification based on a predefined cut-off perfusion value is significantly limited, since it assumes that a single lumped parameter is capable of predicting risk levels of infarction. Physiologically, this is likely not the case, which might explain the significant discrepancies in prediction and actual patient outcomes. Here, we take a probabilistic approach to overcome the limitations of conventional univariate regression methods, by incorporating continuous time-dependent and space-dependent volumetric perfusion maps directly into probabilities for prediction of infarct volumes without applying a threshold. Another advantage of our machine learning approach over conventional regression approaches is that we can incorporate heterogeneous sources of information using a probabilistic mixture scheme. In addition to perfusion measures, we can include other physiological and sociodemographic factors to improve the model's performance.

We employ a probabilistic data-driven model to determine the health status of all tissue voxels and to build data-driven biofidelic predictors for tissue viability. First, we separate tissue regions into infarct and penumbra based on real final tissue outcome from our existing follow-up images, which are co-registered with the CTA source images. Blinded to the numerical results, while overseeing the classification process, we randomly inspected a quarter of the images to ensure clinical fidelity. We used the simulated fluid flow data to determine tissue infarction and penumbra, and compared against current definitions of tissue viability based on CBF and CBV. For each voxel, we use continuous temporal profiles as input and determine the health status of the tissue as an output to train our Binary Gaussian Process (BGP) classifier, which we have previously used to determine flow characteristics in other fluid dynamics systems. Voxel-wise perfusion values are converted to voxel-wise infarct probability by BGP analysis. The expected infarct volume are then calculated as the cumulative sum of infarct probabilities across all voxels. We cross-validate these machine learning predictors and their associated uncertainties by splitting our simulated cases into distinct training (75%) and trial (25%) groups. We compute the mean and variance of the performance of the trial set and compare against the predictors trained using the training dataset. To train the classifier, we use two sources of input data. First, raw 3D perfusion measures from CFD simulations (CBF, CBV, MTT, TTP) and their mathematical time-domain characteristics (peak value, duration, change in time) as well as frequency-domain characteristics (power-spectral density and wavelet transform basis functions) are used. Secondly, we include the currently used NIH Stroke Scale (NUBS) gold standard score, and other non-hemodynamic factors (age, gender, history of diabetes, and hypertension) into our data-driven model. As such, we are capable of integrating the current clinical practice and other factors likely to impact collateral flow (age and hypertension) into our predictor estimates and provide a rich informational toolbox for clinical use.

To identify potential distinguishing features between infarct and penumbra, and considering that features may not be normally distributed, we perform a two-sided unpaired Wilcoxon Rank-Sum test between the distributions of each feature, and adjust comparisons using the Bonferroni correction method to judge statistical significance (p-value threshold of 0.001). As the number of features is larger than the number of training cases, we perform principal component analysis (PCA) on the standardized (zero mean, unit variance) feature matrix to avoid the anticipated redundancy in the feature set, as we previously showed for a similarly large feature set. We extract the best set of features and the associated level of fidelity through uncertainty quantification. These parameters optimize the probabilistic BGP classifier, using the cost functions (1) area under the receiver operating characteristics (ROC) curve (AUC), and (2) the F-measure, which is the harmonic mean of sensitivity and precision. Toavoid over-fitting, these cost functions may be calculated from leave-one-out cross validation, instead of calculating the measures from training. By using time-dependent perfusion calculations instead of the lumped quantities as input, our machine learning algorithm can outperform currently available parameters for stroke assessment.

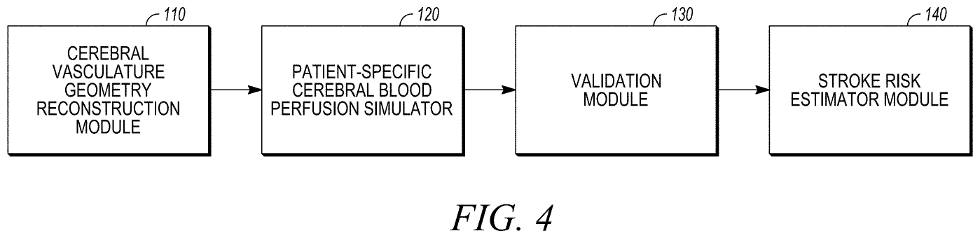

FIG. 4 is functional block diagram illustrating one example of a system for modeling and predicting stroke severity. The system includes a cerebral vasculature geometry reconstruction module 110, a patient-specific cerebral blood perfusion simulator 120, a validation module 130, and a stroke risk estimator module 140. The cerebral vasculature geometry reconstruction module 110 reconstructs the geometry of the cerebral vasculature from medical imaging (e.g., computed tomography angiography images). The patient-specific cerebral blood perfusion simulator 120 simulates patient-specific cerebral blood perfusion in real-time using high-performance computing. The validation module 130 validates the results of numerical simulation with CT perfusion measurements. The stroke risk estimator module 140 develops stroke risk estimates based on patient data and ABCD2 standard.

It should be noted that while the imaging technique described above that is used in developing the simulations by reconstructing the geometry of the cerebral vasculature is CTA, more generally any angiography type imaging technique may be employed, including, without limitation, magnetic resonance angiography (MRA) and ultrasound.

Based upon a rigorous mathematical approach and, in particular, the emphasis on validation against a large clinical dataset, our results present significant refinement over existing modeling approaches for stroke severity assessment and tissue viability. The 3D visualizations from CTA reconstructed vessel geometries could be readily used in the clinic for better demonstration of sites of occlusion and adjacent collateral vessel density distribution. In light of positive results from recent clinical trials and the likely increase in perfusion-based therapy selection criteria, our 3D perfusion maps could provide invaluable information in terms of stroke severity and could be used in place of perfusion scans if unavailable. Finally, the resulting high-fidelity predictors are able to automatically distinguish infarct tissue from salvageable penumbra and help improve the decision-making process for path of treatment.

The various diagrams illustrating various embodiments may depict an example architectural or other configuration for the various embodiments, which is done to aid in understanding the features and functionality that can be included in those embodiments. The present disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement various embodiments. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

It should be understood that the various features, aspects and/or functionality described in one or more of the individual embodiments are not limited in their applicability to the particular embodiment with which they are described, but instead can be applied, alone or in various combinations, to one or more of the other embodiments, whether or not such embodiments are described and whether or not such features, aspects and/or functionality is presented as being a part of a described embodiment. Thus, the breadth and scope of the present disclosure should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

Moreover, various embodiments described herein may be described in the general context of method steps or processes, which may be implemented in one embodiment by a computer program product, embodied in, e.g., a non-transitory computer-readable memory, including computer-executable instructions, such as program code, executed by computers in networked environments. A computer-readable memory may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

As used herein, the term module can describe a given unit of functionality that can be performed in accordance with one or more embodiments. As used herein, a module might be implemented utilizing any form of hardware, software, or a combination thereof. For example, one or more processors, controllers, ASICs, PLAs, PALs, CPLDs, FPGAs, logical components, software routines or other mechanisms might be implemented to make up a module. In implementation, the various modules described herein might be implemented as discrete modules or the functions and features described can be shared in part or in total among one or more modules. In other words, as would be apparent to one of ordinary skill in the art after reading this description, the various features and functionality described herein may be implemented in any given application and can be implemented in one or more separate or shared modules in various combinations and permutations. Even though various features or elements of functionality may be individually described or claimed as separate modules, one of ordinary skill in the art will understand that these features and functionality can be shared among one or more common software and hardware elements, and such description shall not require or imply that separate hardware or software components are used to implement such features or functionality. Where components or modules of the invention are implemented in whole or in part using software, in one embodiment, these software elements can be implemented to operate with a computing or processing module capable of carrying out the functionality described with respect thereto. The presence of broadening words and phrases such as “one or more,” “at least,” “but not limited to” or other like phrases in some instances shall not be read to mean that the narrower case is intended or required in instances where such broadening phrases may be absent.

1. A method for obtaining information for use in determining stroke severity, comprising:
simulating, with a processor executing instructions stored on a compute readable medium, cerebral blood flow (CBF) using as inputs information extracted from an angiography scan of the stroke patient and information concerning blood flow in cranial arteries; and
assessing cerebral tissue viability of the stroke patient based at least in part on the simulated CBF.

2. The method of claim 1, further comprising extracting three-dimensional (3D) vascular geometry information from the angiography scan, the vascular geometry information being one of the inputs used to simulate the CBF of the stroke patient.

3. The method of claim 1, wherein the information concerning blood flow in the cranial arteries includes generic, non-patient specific cranial arterial information.

4. The method of claim 1, wherein the information concerning blood flow in the cranial arteries includes patient-specific cranial arterial information.

5. The method of claim 4, further comprising obtaining the patient-specific cranial arterial information from Doppler ultrasound and/or pressure sensors applied to the neck of the stroke patient.

6. The method of claim 1, wherein assessing tissue viability includes generating a time and space dependent volumetric perfusion map and risk estimates for tissue viability.

7. The method of claim 6, further comprising predicting tissue infarction and penumbra from the perfusion map using a classifier trained with machine-learning techniques.

8. The method of claim 1, wherein simulating CBF of the stroke patient uses a one-dimensional piping model that accounts for viscoelastic compliance of arterial walls.

9. The method of claim 8, further comprising performing the CBF simulation using parallel processing and numerical discretization based on a high order Discontinuous-Galerkin method to achieve a simulation in near-real time.

10. The method of claim 1, wherein the simulating and accessing are conducted by a medical emergency responder prior to transport of the stroke patient to a hospital or other medical treatment facility.

11. The method of claim 2, wherein extracting the 3D vascular geometry information includes applying a vessel-enhancement filter to calculate intensity and curvature in each voxel of the angiography scan.

12. The method of claim 11, wherein extracting the 3D vascular geometry further comprises calculating blood vessel diameter, length and tortuousity and a connectivity matrix describing branching patterns.

13. The method of claim 10, further comprising performing the angiography scan, the angiography scan being performed by the medical emergency responder prior to transport of the stroke patient to the hospital or other medical treatment facility.

14. The method of claim 9, further comprising assigning non-overlapping brain vascular domains to different processors for processing the non-overlapping brain vascular domains in parallel.

15. The method of claim 1, wherein the angiography scan is a computed-tomography scan.

16. The method of claim 1, wherein the angiography scan is a magnetic resonance angiography (MRA) scan.

17. The method of claim 1, wherein the angiography scan is an ultrasound scan.

18. A kit comprising a portable medical device that performs the method of claim 1.
